(19) United States
(12) Patent Application Publication
(10) Pub. No.: US 2003/0134329 A1
(43) Pub. Date: Jul. 17, 2003

(54) Cross-species bioactive peptides

(76) Inventors: Thea Norman, Belmont, MA (US); Sofie R. Salama, Santa Cruz, CA (US); Joshua Trueheart, Concord, MA (US); G. Todd Milne, Brookline, MA (US); Peter Hecht, Newton, MA (US)

Correspondence Address: FISH & RICHARDSON PC, 225 FRANKLIN ST, BOSTON, MA, 02110, US

(21) Appl. No.: 10/267,251

(22) Filed: Oct. 9, 2002

Related U.S. Application Data
(60) Provisional application No. 60/328,340, filed on Oct. 9, 2001.

(51) Int. Cl. G01N 33/53; C12N 1/18; C12N 15/85; C12N 15/74; C12N 5/06

(52) U.S. Cl. 435/7.1; 435/455; 435/325; 435/254.2; 435/483

(57) ABSTRACT

The invention relates to the modulation of fungal signaling pathways. More particularly, the invention relates to compounds that modulate such pathways and to methods for identifying such compounds. The invention provides novel modulators of fungal gene expression that can be used to regulate or modulate activities of specific signaling pathways and to identify and validate potential targets in drug discovery efforts.

This application claims the benefit of prior U.S. provisional application Serial No. 60/328,340, filed Oct. 9, 2001.

### BACKGROUND OF THE INVENTION

1. Field of the Invention

The invention relates to the modulation of fungal signaling pathways. More particularly, the invention relates to compounds with cross-species bioactivity for modulating such pathways, and to methods for identifying such compounds.

2. Summary of the Related Art

Modulation of signaling pathways in fungi can have important effects on the production of primary and secondary metabolites by production strains of fungi, as well as the pathogenicity of disease-causing fungi.

Secondary metabolite production by fungi has been an extremely important source of a variety of therapeutically significant pharmaceuticals. β-lactam antibacterials such as penicillin and cephalosporin are produced by Penicillium chrysogenum and Acremonium chrysogenum, respectively, and these compounds are by far the most frequently used antibacterials (reviewed in Luengo and Penalva (1994), Prog. Ind. Microbiol. 29: 603-38; Jensen and Demain (1995), Biotechnology 28: 239-68; Brakhage (1998), Microbiol. Mol. Biol. Rev. 62: 547-85). Cyclosporin A, a member of a class of cyclic undecapeptides, is produced by Tolypocladium inflatum. Cyclosporin A dramatically reduces morbidity and increases survival rates in transplant patients (Borel (1986), Prog. Allergy 38: 9-18). In addition, several fungal secondary metabolites are cholesterol lowering drugs, including lovastatin, which is made by Aspergillus terreus and several other fungi (Alberts et al. (1980), Proc. Natl. Acad. Sci. USA 77: 3957-3961). These and many other fungal secondary metabolites have contributed greatly to health care throughout the world (see Demain (1992), Ciba Found. Symp. 171: 3-16; Bentley (1999), Crit. Rev. Biotechnol. 19: 1-40).

Unfortunately, many challenges are encountered between the discovery of a useful secondary metabolite activity and the production of significant quantities of pure drug. Thus, various efforts have been made to improve the production of commercially significant secondary metabolites by fungi. Some of these efforts have attempted to improve production by modification of the growth medium or the bioreactor used to carry out the fermentation. Buckland et al. (1989), in Topics in Industrial Microbiology: Novel Microbial Products for Medicine and Agriculture, pp. 161-169, Elsevier, Amsterdam, discloses improved lovastatin production by modification of the carbon source, and also teaches the superiority of a hydrofoil axial flow impeller in the bioreactor. Other efforts have involved strain improvements, either through re-isolation or random mutagenesis. Agathos et al. (1986), J. Ind. Microbiol. 1: 39-48, teaches that strain improvement and process development together resulted in a ten-fold increase in cyclosporin A production.

More recently, strains have been improved by manipulation of the genes encoding the biosynthetic enzymes that catalyze the reactions required for production of secondary metabolites. Penalva et al. (1998), Trends Biotechnol. 16: 483-489, discloses that production strains of P. chrysogenum have increased copy number of the penicillin synthesis structural genes. Other studies have modulated expression of other biosynthetic enzyme-encoding genes, thereby affecting overall metabolism in the fungus. Mingo et al. (1999), J. Biol. Chem. 21: 14545-14550, demonstrate that disruption of phacA, a gene required for phenylacetate catabolism in Aspergillus nidulans, leads to increased penicillin production, probably by allowing increased availability of phenylacetate for secondary metabolism. Similarly, disruption of the gene encoding aminoadipate reductase in P. chrysogenum increased penicillin production, presumably by eliminating competition for the substrate α-aminoadipate (Casquiero et al. (1999), J. Bacteriol. 181: 1181-1188).

However, only a limited number of biosynthetic genes have been identified, and the role of some of these genes is not yet clear. There is, therefore, a need for new types of modulators that can affect the signaling pathways involved in the production of primary and secondary metabolites. These new modulators could be potentially important tools to improve the production of these primary or secondary metabolites.

There is also a need for new modulators of fungal virulence. Fungal infections are a serious health concern, especially in immunocompromised patients. Ha and White (1999), Antimicrobial Agents and Chemotherapy 43: 763-768, teaches that candidiasis, which is caused by the pathogenic yeast Candida albicans, is the most frequent fungal infection associated with AIDS and other immunocompromised states. Weig et al. (1998), Trends in Microbiology 6: 468-470, discloses that the frequency of Candida infections has increased in recent years and has been accompanied by a significant rise in morbidity and mortality.

Recently, there has been great interest in identifying genes that may be implicated as important virulence factors in these infections. Calera et al. (2000), Infection and Immunity 68: 518-525, discloses that the SSK1 response regulator gene from C. albicans is essential for normal hyphal development and virulence. Alex et al. (1998), Proc. Natl. Acad. Sci. USA 95: 7069-7073, teaches that COS1, a two-component histidine kinase, is required for normal hyphal growth of C. albicans, and may play a role in the virulence properties of the organism. Alonso-Monge et al. (1999), J. Bacteriology 181: 3058-3068, teaches that deletion of the C. albicans gene encoding the mitogen-activated protein kinase HOG1 causes derepression in serum induced hyphal formation and a dramatic increase in the survival time of systemically infected mice. Csank et al. (1998), Infection and Immunity 66: 2713-2721, discloses that disruption of the C. albicans mitogen activated protein kinase CEK1 adversely affects the growth of serum induced mycelial colonies and attenuates virulence in a mouse model for systemic candidiasis. These and other studies have suggested that hyphal growth may be an important virulence factor in C. albicans. Lo et al. (1997), Cell 90: 939-949, teaches that nonfilamentous C. albicans mutants are avirulent.

### BRIEF SUMMARY OF THE INVENTION

The invention relates to the identification of peptides that are cross-species bioactive in their ability to modulate specific fungal signaling pathways. More particularly, the invention relates to both methods of identifying bioactive peptides that modulate such pathways and to the bioactive peptides themselves. Thus, the invention provides novel modulators of fungal gene expression that can be used to regulate or modulate the activities of specific signaling pathways and to identify and validate potential targets in drug discovery efforts.

Thus, in a first aspect, the invention provides methods for identifying cross-species bioactive peptides, the methods comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype. Optionally, the methods may further include the steps of choosing a fungus of the second species having the second phenotype, identifying the bioactive peptide in the chosen fungus of the second fungal species, and determining whether the bioactive peptides identified from the first species and the second species are the same. The first phenotype of the first fungal species and the first phenotype of the second fungal species may be the same or different. The second phenotype of the first fungal species and the second phenotype of the second fungal species may be the same or different. In each embodiment, the phenotypes may relate to the production of primary or secondary metabolites or enzymes, or to virulence.

In a second aspect, the invention provides cross-species bioactive peptides identified by the methods according to the first aspect of the invention.

In a third aspect, the invention provides peptidomimetics of cross-species bioactive peptides identified by the methods according to the first aspect of the invention.

In a fourth aspect, the invention provides a fungus of the second species expressing the cross-species bioactive peptide according to the first aspect of the invention.

In a fifth aspect, the invention provides methods for producing a primary or secondary metabolite, the methods comprising culturing the fungus according to the fourth aspect under conditions that allow production of the primary or secondary metabolite.

In a sixth aspect, the invention provides methods for producing an enzyme, the methods comprising culturing the fungus according to the fourth aspect under conditions that allow production of the enzyme.

In a seventh aspect, the invention provides methods for identifying a genetic target responsible for fungal virulence, the methods comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the first or second phenotype of the second species. In this aspect, the difference between the first and second phenotypes of the second species relate to virulence.

In an eighth aspect, the invention provides cross-species bioactive peptides identified by the methods according to the seventh aspect of the invention.

In a ninth aspect, the invention provides peptidomimetics of the cross-species bioactive peptides identified by the methods according to the seventh aspect of the invention

In a tenth aspect, the invention provides a fungus of the second species expressing the cross-species bioactive peptide identified by the methods according to the seventh aspect of the invention.

In an eleventh aspect, the invention provides methods for identifying a genetic target responsible for modulating primary metabolite production, the methods comprising expressing in a first fungal species having a first phenotype a bioactive peptide to yield a second phenotype, choosing a fungus of the first species having the second phenotype, expressing the bioactive peptide in a second fungal species having a first phenotype to yield a second phenotype, choosing a fungus of the second species having the second phenotype, and identifying a target responsible for the first or second phenotype of the second fungal species. In this aspect, the difference between the first and second phenotypes of the second species relate to primary metabolite production.

In a twelfth aspect, the invention provides methods for identifying a genetic target responsible for modulating secondary metabolite production, the methods comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the first or second phenotype of the second fungal species. In this aspect, the difference between the first and second phenotypes of the second species relate to secondary metabolite production.

In a thirteenth aspect, the invention provides methods for identifying a genetic target responsible for modulating enzyme production, the methods comprising expressing in a first fungal species having a first phenotype a bioactive peptide to yield a second phenotype, choosing a fungus of the first species having the second phenotype, identifying the bioactive peptide in the chosen fungus of the first fungal species expressing the bioactive peptide in a second fungal species having a first phenotype to yield a second phenotype, choosing a fungus of the second species having the second phenotype, and identifying a target responsible for the first or second phenotype of the second fungal species. In this aspect, the difference between the first and second phenotypes of the second species relate to production of the relevant enzyme.

In a fourteenth aspect, the invention provides methods for identifying a genetic target responsible for modulating a particular characteristic of a fungus, the methods comprising expressing in a first fungal species having a first phenotype a bioactive peptide to yield a second phenotype, choosing a fungus of the first species having the second phenotype, identifying the bioactive peptide in the chosen fungus of the first fungal species expressing the bioactive peptide in a second fungal species having a first phenotype to yield a second phenotype, choosing a fungus of the second species having the second phenotype, and identifying a target responsible for the first or second phenotype of the second fungal species. In this aspect, the difference between the first and second phenotypes of the second species relate to the particular characteristic.

In a fifteenth aspect, the invention features a method for identifying cross-species bioactive peptides, comprising:

providing cells of a first species having a first phenotype;

transforming the cells of a first species having a first phenotype with a nucleic acid molecule encoding a polypeptide comprising a random peptide sequence to provide a library of cells of the first species expressing a polypeptide comprising a random peptide sequence;

selecting from the library of cells of the first species a cell having a second phenotype;

identifying the random peptide sequence expressed by the selected cell as a bioactive polypeptide;

expressing the bioactive polypeptide in a cell of a second species to produce a cell of a second species having the second phenotype; and

identifying a bioactive peptide that when expressed in the cell of the second species produces a cell of the second species having a second phenotype as a cross-species bioactive peptide.

The polypeptide expressed by the cell is preferably identical in all library members except for the random peptide. In various embodiments the random peptide portion consists of between 5 and 50 amino acids, between 5 and 40 amino acids, between 10 and 30 amino acids, and between 10 and 30 amino acids.

In various preferred embodiments, the first and second species are selected from the group consisting of mammalian species, the first and second species are selected from the group consisting of mammalian species, insect species (e.g., D. melanogaster), bacteria species (e.g., E. coli, Staphylococcus ssp, Enterococcus faecalis, Pseudomonas, auerigonosa), plant species, and fungal species, the first and second species are fungal species, the polypeptide does not comprise either a DNA binding domain or a transcription activation domain, the first species is selected from the group consisting of Saccharomyces cerevisiae, Aspergillus nidulans, Candida sp., and Neurospora crassa, the second species is selected from the group consisting of Candida sp., Aspergillus sp., Penicillium sp., Acremonium chrysogenum, Yarrowia lipolytica, Phaffia rhodozyma, Mucor sp., Rhizopus sp., Fusarium sp., Penicillium marneffei, Microsporum sp., Cryptococcus neoformans, Pneumocystis carinii, Trichophyton sp., and Ustilago maydis, Nodulisporium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Neurospora sp., Pestalotiopsis sp. and Phaffia rhodozyma, the cells of the first species are wild-type, the second phenotype of the first fungal species is selected from the group consisting of modulated expression of S. cerevisiae FLO11 or SMP1, modulated invasion, modulated colony morphology, modulated adherence to solid substrate, modulated pseudohyphal growth, modulated expression of a FLO11 reporter gene, and modulated expression of a SMP1 reporter gene, the first phenotype of the second fungal species is selected from the group consisting of altered virulence, avirulent, and hypervirulent, the second phenotype of the second fungal species is selected from the group consisting of modulated production of a metabolite or enzyme, increased storage viability, altered cell or colony morphology, altered temperature tolerance, conditional cell death, growth inhibition, conditional lysis, resistance to deleterious effects of exposure to a primary or secondary metabolite or enzyme, hypervirulence, avirulence, increased pathogenesis, decreased pathogenesis, altered adherence characteristics, and modulated expression of an HWP1-reporter gene

In a sixteenth aspect, the invention features a cross-species bioactive peptide comprising an amino acid sequence comprising, consisting of, or consisting essentially an amino acid sequence selected from the group consisting of: SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 1, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, and SEQ ID NO. 33

In an seventeenth aspect the invention features method for producing a secondary metabolite, a primary metabolite or an enzyme by culturing a fungal cell expressing a cross-species bioactive peptide under conditions suitable for production of the secondary metabolite, the primary metabolite or the enzyme.

In an eighteenth aspect, the invention features peptidomimetics of a bioactive peptide identified by expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype.

In a nineteenth aspect, the invention features a method for identifying a cross-species peptide-interacting polypeptide that binds to a cross-species bioactive peptide, comprising:

providing cells of a first species having a first phenotype;

transforming the cells of a first species having a first phenotype with a nucleic acid molecule encoding a polypeptide comprising a random peptide sequence to provide a library of cells of the first species expressing a polypeptide comprising a random peptide sequence;

selecting from the library of cells of the first species a cell having a second phenotype;

identifying the random peptide sequence expressed by the selected cell as a bioactive polypeptide;

expressing the bioactive polypeptide in a cell of a second species to produce a cell of a second species having the second phenotype;

identifying a bioactive peptide that when expressed in the cell of the second species produces a cell of the second species having a second phenotype as a cross-species bioactive peptide;

contacting the cross-species bioactive peptide with a test polypeptide to determine if the test polypeptide interacts with the cross-species bioactive peptide; and

identifying the test polypeptide as a cross-species peptide-interacting polypeptide if the test polypeptide binds to the cross-species bioactive peptide.

### DETAILED DESCRIPTION

The invention relates to the identification of peptides that are cross-species bioactive in their ability to modulate, for example, specific fungal signaling pathways. More particularly, the invention relates to both methods of identifying bioactive peptides that modulate such pathways and to the bioactive peptides themselves. Thus, the invention provides novel modulators of gene expression that can be used to regulate or modulate the activities of specific signaling pathways and to identify and validate potential targets in drug discovery efforts.

The methods of the invention are useful for identifying peptides that are active in two different species, e.g., a first fungal species and a second fungal species or a fungal species and a mammalian species or a fungal species and a mammalian species.

The cross-species bioactive peptides may act in a number of ways. For example, but not by way of limitation, a bioactive peptide may act by directly interfering with or substituting for the function of a wild-type protein containing the peptide sequence or by inhibiting or activating the function of proteins in a pathway through direct protein-protein interactions.

The patent, scientific and medical publications referred to herein establish knowledge that was available to those of ordinary skill in the art at the time the invention was made. The entire disclosures of the issued U.S. patents, published and pending patent applications, and other references cited herein are hereby incorporated by reference.

Definitions

In order to more clearly and concisely describe the subject matter which is the invention, the following definitions are provided for certain terms which are used in the specification and appended claims.

As used herein, the term “bioactive peptide” means a peptide that confers an altered phenotype on an organism. Preferred bioactive peptides according to the invention have from about 4 to about 16 amino acids. In certain preferred embodiments, the candidate bioactive peptides according to the invention may comprise a portion of a fusion protein in which the candidate bioactive peptide is fused with a protein scaffold, such as the Staphylococcal nuclease scaffold disclosed in Norman et al. (1999), Science 285:591-595. In cases in which a portion of the intended scaffold protein enhances the bioactivity of the intended candidate bioactive peptide, that portion of the intended scaffold protein is considered part of the bioactive peptide.

As used herein, the term “identifying a bioactive peptide” means physically or chemically isolating or locating a peptide such that the peptide is available, or can readily be made available, for further use or analysis. Once a bioactive peptide is identified, the amino acid sequence of the bioactive peptide can be determined. However, a peptide need not be actually sequenced in order to be identified. Thus, for example, peptides obtained from libraries of peptides may be identified without being sequenced if the clone containing the peptide is determined and the peptide could be sequenced later. In the examples below, the sequence of bioactive peptides may be determined by sequencing the encoding DNA using primers complementary to the sequences of the scaffold flanking the peptide. In other cases, similar flanking sequences can be used for sequencing.

As used herein, the term “expressing a bioactive peptide” means transforming a plurality of fungi with a plurality of peptides, some of which are bioactive, and allowing such bioactive peptide to be expressed in a fungus transformed with the bioactive peptide.

As used herein, the term “modulated production” of a primary or secondary metabolite or enzyme means a positive or desirable change in one or more of the variables or values that affect the process or results of production of the metabolite or enzyme in a liquid or solid state fungal fermentation. For example, in the case where the modulation was an increased production of metabolite or enzyme, these positive or desirable changes include, without limitation, an increase in the amount of the metabolite or enzyme being produced (in absolute terms or in quantity per unit volume of fermentation broth or per unit mass of solid substrate); a decrease in the volume of the broth or the mass/quantity of substrate required for the production of sufficient quantities; a decrease in the cost of raw materials and energy, the time of fermentor or culture run, or the amount of waste that must be processed after a fermentor run; an increase in the specific production of the desired metabolite or enzyme (both in total amounts and as a fraction of all metabolites and proteins and side products made by the fungus); an increase in the percent of the produced metabolite or enzyme that can be recovered from the fermentation broth or culture; and an increase in the resistance of an organism producing a metabolite or enzyme to possible deleterious effects of contact with the metabolite or enzyme or increased levels of the metabolite or enzyme. As will be apparent to those of ordinary skill in the art, in some instances, the desired modulation will be a decrease in production of a metabolite or enzyme. In those cases, positive or desirable changes in the variables or values that affect the process or results of production of the metabolite or enzyme will be those which lead to decreased production or activity.

As used herein, the term “primary metabolite” means a natural product that has a central or necessary role in the normal functioning of an organism. Primary metabolites are compounds that are normally involved in primary metabolic processes such as cell respiration, growth or division, and include, without limitation, compounds involved in the biosynthesis of lipids, carbohydrates, proteins, and nucleic acids.

As used herein, the term “secondary metabolite” means a compound that is a natural product of an organism but is not normally involved in primary metabolic processes such as cell respiration, growth or division, and is not required for growth under standard conditions. Secondary metabolites do not include ethanol or fusel alcohols. Secondary metabolites can be derived from intermediates of many pathways of primary metabolism. These pathways include, without limitation, pathways for the biosynthesis of amino acids, the shikimic acid pathway for biosynthesis of aromatic amino acids, the polyketide biosynthetic pathway from acetyl coenzyme A (CoA), the mevalonic acid pathway from acetyl CoA, and pathways for the biosynthesis of polysaccharides and peptidopolysaccharides. (See, e.g., Griffin, ed., (1994), Fungal Physiology, Chapter 9, John Wiley & Sons, Inc., New York, pp 246-274) “Secondary metabolites” also include intermediate compounds in the biosynthetic pathway for a secondary metabolite that are dedicated to the pathway for synthesis of the secondary metabolite. “Dedicated to the pathway for synthesis of the secondary metabolite” means that once the intermediate is synthesized by the cell, the cell will not convert the intermediate to a primary metabolite. “Intermediate compounds” also include secondary metabolite intermediate compounds which can be converted to useful compounds by subsequent chemical conversion or subsequent biotransformation. As such, providing improved availability of such intermediate compounds can improve the production of the ultimate useful compound, which itself may be referred to herein as a secondary metabolite.

As used herein, an “increase” or “decrease” in any variable or value means a statistically significant increase or decrease, as measured by an means generally accepted in the art for measuring the relevant variable or value.

As used herein with respect to fungal strains and with respect to a trait of interest, the term “wild-type” means presenting phenotypic features common to the majority of the strains that can be isolated in nature. A wild-type strain must not be mutated with respect to the trait of interest, such that it presents a null, significantly deleterious or significantly abnormal phenotype for that trait. However, with respect to other traits, wild-type strains can present uncommon phenotypes. For example, a wild-type strain can be abnormally auxotrophic for certain compounds, or abnormally resistant or sensitive to certain compounds, in order to use these traits as selectable marker systems. For example, an S. cerevisiae strain with ura3-52, trp1-1 and leu2 auxotrophies would still be considered wild-type if the trait of interest were FLO11 expression.

As used herein, a “gene responsible for a phenotype” means a gene whose expression is necessary or sufficient for the phenotype to be present or expressed, or which affects the degree or nature of expression of the phenotype. As used herein, the term “genetic target responsible for a phenotype” means a protein, peptide, carbohydrate, lipid or nucleic acid which is necessary or sufficient for the phenotype to be present or expressed, or which affects the degree or nature of expression of the phenotype. In these contexts, relevant phenotypes can include, without limitation, virulence, avirulence, pathogenesis, production of a primary or secondary metabolite, production of an enzyme, storage viability, temperature tolerance, cell or colony morphology, invasion characteristics, adherence characteristics, growth characteristics, growth inhibition, conditional cell death, conditional lysis, expression of a gene or protein, or resistance to the deleterious effects of a primary or secondary metabolite or enzyme.

A polypeptide that contains a random peptide is a polypeptide, e.g., a naturally occurring protein that includes a random peptide sequence insert. The insert can replace a portion of the naturally-occurring polypeptide.

General Considerations

In general, the present invention provides methods for rapidly identifying cross-species bioactive peptides. Candidates for cross-species bioactive peptides are identified in a first species by their ability to cause a phenotypic change in that species, and then identified as cross-species bioactive by testing their ability to cause a phenotypic change in a second species. In some embodiments, the first species is a well-characterized model system, and the second species is a less tractable organism of interest. Characteristics of less tractable systems often include less robust molecular tools (e.g., poor transformation efficiency) and strain traits (e.g., growth morphology) that are not desirable for screening. Therefore, the invention provides a rapid strategy for identifying bioactive peptides via genetic selection in a more tractable organism, e.g. Saccharomyces cerevisiae, and testing them for cross-species activity in related fungi. This strategy is made possible by conserved signaling pathways amongst even distantly related fungi. These cross-species bioactive peptides can serve as valuable tools to identify and validate protein targets in pathogenic fungi and to impact the production of pharmaceuticals and commodities, including primary and secondary metabolites and enzymes, produced in industrial fungal fermentations.

Thus, in a first aspect, the invention provides methods for identifying cross-species bioactive peptides, the methods comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second fungal species, and identifying the bioactive peptide in the chosen fungus of the second fungal species. The first phenotype of the first fungal species and the first phenotype of the second fungal species may be the same or different. The second phenotype of the first fungal species and the second phenotype of the second fungal species may be the same or different.

In certain preferred embodiments, the first fungal species is selected from the group consisting of S. cerevisiae, Candida sp., A. nidulans, and Neurospora crassa. In certain embodiments, the second fungal species is selected from the group including, without limitation Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp. and Phaffia rhodozyma. An extensive, but non-limiting, listing of suitable fungi may be found in Chapter 9 (Secondary(Special) Metabolism) of Fungal Physiology, Griffin, David H., John Wiley & Sons, Inc.; ISBN: 0471166154.

In certain preferred embodiments the first phenotype of the first fungal species is wild type with respect to the trait of interest. In certain preferred embodiments, the second phenotype of the first fungal species is selected from the group consisting of modulated expression of FLO11 or SMP1, altered invasion properties, altered colony morphology, increased or decreased adherence to a solid substrate (e.g.,), altered pseudohyphal growth, conditional cell death and inhibition of cell growth.

In certain preferred embodiments the first phenotype of the second fungal species is wild type with respect to the trait of interest. In certain preferred embodiments, the second phenotype of the second fungal species is selected from the group consisting of modulated production of a metabolite or enzyme, increased storage viability, altered cell or colony morphology, altered temperature tolerance, conditional cell death, growth inhibition, conditional lysis, altered expression of an enzyme, or resistance to the deleterious effects of a primary or secondary metabolite or enzyme.

In a second aspect, the invention provides bioactive peptides identified according to the methods of the first aspect of the invention. Such bioactive peptides include, without limitation, the peptides shown in Table 1 below.

TABLE 1

| | | | | FL011- |
|---|---|---|---|---|
| | | P_{FLO11}- | Inva- | lacZ |
| | | neo | sion | (relative |
| Clone | Peptide Sequence | Mediated | Pheno- | expres- |
| Name | (in loop) | G418^{R} | type | sion) |
| 5-28, 3 | GRRSRVRWSWPLFKSL | + | ++ | 18.2 |
| 5-39-1a | QPWYRKLRLAPVYPSD | + | +++ | 17.8 |
| 5-32, 1 | PYGPFLLLTPF*A*L | + | +++ | 15.1 |
| 5-75-1 | ELAYKFGRDWARLYFS | + | +++ | 15.0 |
| 4-16, 2 | KRKVCLLGGRFLVEWL | + | +++ | 14.8 |
| 5-25, 2 | RRWEVLRGDRTARLLS | + | ++ | 13.7 |
| 5-16, 3 | ALARWLEIELHPQGLI | + | +++ | 13.3 |
| 5-48, 2 | AFSRRGRRAWSWPVSN | + | +++ | 12.9 |
| 5-31, 2 | SGDAVMGFLLKCLGLQ | + | +++ | 12.6 |
| 5-23, 1a | GHLWEWSGERWLLRWC | + | +++ | 12.2 |
| 4-9, 3 | GYLRKHRALALGLNLH | + | +++ | 11.5 |
| 4-17, 1 | YEAGVILRACLWAVSG | + | +++ | 10.5 |
| 5-43-3b | RCPVAHTLCWEVAGCT | + | +++ | 9.6 |
| 5-30, 3 | CGLGGCCCEIFWSFSN | + | +++ | 7.3 |
| 5-45, 2 | REINWLRFFRLHKVID | + | +++ | 6.2 |
| 5-37, 1 | LYRAFTWPVAKSLRME | + | ++ | 6.1 |
| 5-14, 3 | WWRPHERFIWAQYYLA | + | +++ | 4.9 |
| 5-34, 3 | EWCEICHRLVWVFCFT | + | +++ | 4.1 |
| 5-11-3a | QRIPLIGGVLFIAWIM | + | +++ | 3.0 |
| 5-20-1a | LDLDRVSVNRCSFMGF | + | +++ | 2.8 |
| 5-18-3b | RRWNRLKSWWPCFREQ | + | + | 2.6 |
| 5-12-1a | HGDGLMRYFWSVAMWT | + | ++ | 2.7 |
| 4-3, 1 | GAGCAPRGCVVWSSLL | + | +++ | 2.5 |
| 5-49, 3 | GSNGCVSVSSSVTSFM | +w | +++ | 2.5 |
| 4-1, 1 | KRGRRRSHSCPSLMTD | + | + | 2.2 |
| 5-64-1 | MGCVKRLLWGCCMKLT | + | ++ | 2.0 |
| pTCN23 | Control | − | − | 1.0 |

In a third aspect, the invention provides peptidomimetics of bioactive peptides identified by the methods according to the first aspect of the invention. Peptides such as those shown in Table 1 can be analyzed for their predicted tertiary structure, based upon which chemically constrained analogs can be made employing conventional procedures or purchased from commercial suppliers, such as Synthetech, Inc. (Albany, Oreg.).

In a fourth aspect, the invention provides a fungus of the second species expressing such a cross-species bioactive peptide. Such fungi can be produced according to standard methods of transforming fungi as known in the art including, but not limited to, the methods disclosed in the examples below.

In a fifth aspect, the invention provides methods for producing a primary or secondary metabolite, the methods comprising culturing a fungus according to the fourth aspect under conditions suitable for production of the primary or secondary metabolite. “Conditions suitable for production of the primary or secondary metabolite” means culture conditions under which the fungus does in fact produce the desired primary or secondary metabolite.

In a sixth aspect, the invention provides methods for producing an enzyme, the methods comprising culturing the fungus according to the fourth aspect under conditions suitable for production of the enzyme. “Conditions suitable for production of the enzyme” means culture conditions under which the fungus does in fact produce the desired enzyme.

In a seventh aspect, the invention provides methods for identifying a genetic target responsible for fungal virulence, the methods comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a genetic target responsible for the first or second phenotype of the second fungal species. The first phenotype of the first fungal species and the first phenotype of the second fungal species may be the same or different. The second phenotype of the first fungal species and the second phenotype of the second fungal species may be the same or different. In these embodiments, at least one of the phenotypes is related to virulence.

In certain preferred embodiments, the first fungal species is selected from the group consisting of S. cerevisiae, A nidulans, Candida sp., and N. crassa. In certain embodiments, the second fungal species is selected from the group consisting of Candida sp., Aspergillus sp., Mucor sp., Rhizopus sp., Fusarium sp., Penicillium marneffei, Microsporum sp., Cryptococcus neoformans, Pneumocystis carinii, Trichophyton sp., and Ustilago maydis.

In certain preferred embodiments the first phenotype of the first fungal species is wild type with respect to the trait of interest. In certain preferred embodiments, the second phenotype of the first fungal species is selected from the group consisting of modulated expression of FLO11 or SMP1, altered invasion properties, altered colony morphology, increased or decreased adherence to a solid substrate (e.g., plastic), and altered pseudohyphal growth.

In certain preferred embodiments, the first phenotype of the second fungal species is selected from the group consisting of wild type, hypervirulent, and avirulent. In certain preferred embodiments, the second phenotype of the second fungal species is selected from the group consisting of wild type, hypervirulent, avirulent, increased pathogenesis, decreased pathogenesis, altered adherence characteristics (i.e., to a plastic substrate or to agar), altered cell or colony morphology characteristics (e.g., filamentation), modified temperature tolerance, conditional cell death, growth inhibition, and modulated expression of an HWP1-reporter gene.

In an eighth aspect, the invention provides bioactive peptides identified by the method according to the seventh aspect of the invention. Such bioactive peptides include, without limitation, the peptides shown in Table 2 below.

TABLE 2

| | | | SMP1- | |
|---|---|---|---|---|
| | | Inva- | lacZ | |
| | | sion | (relative | FLO11-lacZ |
| Clone | Peptide Sequence | Pheno- | expres- | (relative |
| Name | (in loop) | type | sion) | expression) |
| D2a | LGYMRASREMDGWKLL | − | 15.4 | 0.3 |
| D2e | LPYEVSRVCWPRRLGY | − | 14.3 | 0.1 |
| D4a | LGYGEAEREMVTCREH | − | 9.1 | 0.1 |
| B2a | LAYGPERYPRACMRWD | − | 8.9 | 0.2 |
| G2a | LSYMEGPYWHRSFRAV | − | 7.5 | 0.5 |
| B2c-2 | LTGYAKLLVQLSYFAM | − | 6.4 | 0.7 |
| G4a-6 | LPVSYVGQSTSQGPVW | − | 7.8 | 0.4 |
| pTCN23 | Control | + | 1.0 | 1.0 |

In a ninth aspect, the invention provides peptidomimetics of bioactive peptides identified by the methods according to the seventh aspect of the invention. Peptides such as those shown in Table 2 can be analyzed for their predicted tertiary structure, based upon which chemically constrained analogs can be made employing conventional procedures or can be purchased from commercial suppliers, such as Synthetech, Inc. (Albany, Oreg.).

In a tenth aspect, the invention provides a fungus of the second species expressing the cross-species bioactive peptides of the invention. Such fungi can be produced according to standard methods of transforming fungi as known in the art including, but not limited to, the methods disclosed in the examples below.

In an eleventh aspect, the invention provides methods for identifying a genetic target responsible for modulating primary metabolite production, the methods comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the second phenotype of the second fungal species. The first phenotype of the first fungal species and the first phenotype of the second fungal species may be the same or different. The second phenotype of the first fungal species and the second phenotype of the second fungal species may be the same or different. In these embodiments, at least one of the phenotypes is related to primary metabolite production.

In a twelfth aspect, the invention provides methods for identifying a genetic target responsible for modulating secondary metabolite production, the methods comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the second phenotype of the second fungal species. The first phenotype of the first fungal species and the first phenotype of the second fungal species may be the same or different. The second phenotype of the first fungal species and the second phenotype of the second fungal species may be the same or different. In these embodiments, at least one of the phenotypes is related to secondary metabolite production.

In a thirteenth aspect, the invention provides methods for identifying a genetic target responsible for modulating enzyme production, the methods comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the first or second phenotype of the second fungal species. The first phenotype of the first fungal species and the first phenotype of the second fungal species may be the same or different. The second phenotype of the first fungal species and the second phenotype of the second fungal species may be the same or different. In these embodiments, at least one of the phenotypes is related to production of the enzyme.

In a fourteenth aspect, the invention provides methods for identifying a genetic target responsible for modulating a particular characteristic of a fungus, the methods comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the first or second phenotype of the second fungal species, wherein the particular characteristic is selected from the group consisting of storage viability, altered cell morphology, altered colony morphology, temperature tolerance, conditional cell death properties, growth inhibition, conditional lysis, and resistance to primary or secondary metabolite toxicity. In these embodiments, at least one of the phenotypes is related to the particular characteristic of the fungus.

With respect to the above-described aspects in which genetic targets are identified (i.e., the seventh, eleventh, twelfth, thirteenth, and fourteenth aspects), the identification of the genetic targets of cross-species bioactive peptides provides insight into specific pathways, proteins, and genes whose activity can be modulated to impact important cellular processes (e.g., metabolite or enzyme production, virulence). The information gathered during target identification can be useful for configuring additional screens or selections for new modulators of metabolite or enzyme production. Similarly, the identification of fungal virulence related targets is useful to developing drug discovery assays and screens. Target identification ultimately requires defining direct or indirect physical interaction between a bioactive peptide and a genetic target. Physical interaction can be identified through various procedures familiar to those skilled in the art, including, without limitation, affinity chromatography followed by mass spectrometry (or other means of specific identification), co-immunoprecipitation, yeast two-hybrid analysis, or protein arrays. In addition, tools such as genetic (i.e., suppressor screens) and microarray analysis are useful methods to identify pathways that bioactive peptides may be modulating, and these technologies can facilitate the target identification process.

Once a cross-species bioactive peptide has been identified, one can identify polypeptides and other cellular components that interact with the peptide. In this manner genetic targets can be identified. Among the methods that can be used for identifying interacting polypeptides are the two-hybrid assay, which assay can be performed in yeast or other cell types. The two-hybrid assay and variations on the two-hybrid assay are known to those skilled in the art (see, e.g., Louvet et al. (1997) Biotechniques 23:816-18). Other methods include co-immunization and co-localization assays (Wong et al. (1997) Anal. Biochem. 252:33-39). Moreover, commercially available kits are available for conducting both tow hybrid assays and co-immunization and co-localization assays. One can also use so-called protein arrays to identify interacting proteins (see PCT Publication No. 01/83827).

The invention features methods for identifying polypeptides or other agents, e.g., cellular components that interact with a cross-species bioactive peptide. In one embodiment, an assay is one in which a cross-species peptide, or a biologically active portion thereof, is contacted with a test compound, e.g., a test polypeptide, and the ability of the test compound to bind to the cross-species peptide determined. Determining the ability of the test compound to bind to the cross-species peptide can be accomplished, for example, by coupling the test compound with a radioisotope or enzymatic label such that binding of the test compound to the cross-species peptide or biologically active portion thereof can be determined by detecting the labeled compound in a complex. For example, test compounds can be labeled with ¹²⁵I, ³⁵S, ¹⁴C, or ³H, either directly or indirectly. Alternatively, test compound can be enzymatically labeled with, for example, horseradish peroxidase, alkaline phosphatase, or luciferase, and the enzymatic label detected by determination of conversion of an appropriate substrate to product.

The assay can be a cell-free assay in which the cross-species peptide or the candidate interacting polypeptide is attached to a solid support to facilitate separation of complexed from uncomplexed forms of one or both of the agents, as well as to accommodate automation of the assay. For example, glutathione-S-transferase/cross-species bioactive peptide fusion proteins or glutathione-S-transferase/test polypeptide fusion proteins can be adsorbed onto glutathione sepharose beads (Sigma Chemical; St. Louis, Mo.) or glutathione derivatized microtitre plates, which are then combined with the test compound or the test compound and either the non-adsorbed test polypeptide protein or cross-species bioactive peptide, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly. In an alternative embodiment, MYC or HA epitope tagged cross-species bioactive peptide fusion proteins or MYC or HA epitope tagged target fusion polypeptides proteins can be adsorbed onto anti-MYC or anti-HA antibody coated microbeads or onto anti-MYC or anti-HA antibody coated microtitre plates, which are then combined with the test compound or the test compound and either the non-adsorbed target polypeptide or cross-species peptide, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly.

The two-hybrid system is based on the modular nature of most transcription factors, which consist of separable DNA-binding and activation domains. Briefly, the assay utilizes two different DNA constructs. In one construct, the gene that codes for a cross-species bioactive peptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. If the “bait” and the “prey” proteins are able to interact, in vivo, forming a cross-speceis bioactive peptide-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the cross-species bioactive peptide.

The following examples illustrate some preferred modes of practicing the present invention, but are not intended to limit the scope of the claimed invention. Alternative materials and methods may be utilized to obtain similar results. The peptide library used in these examples has been previously described (Norman et al. (1999), Science, 285:591-595).

The cross-species bioactive peptides can be used to identify genetic targets by isolating genetic suppressors. For example, one could transform a cell having a second phenotype and expressing a bioactive peptide responsible for the phenotype with a library of cDNAs. One would then identify cells that reverted to the first phenotype. The cDNA present in these cells is a candidate genetic suppressor. Alternatively, the cell expressing the bioactive peptide can be subjected to mutation, e.g., insertional mutation. Revertants to the first phenotype are selected, and the gene having the insertion mutation is identified as a candidate suppressor gene.

### EXAMPLE 1

Selection of Peptides that Promote Expression of FLO11 and Promote Invasion.

To identify peptides that promote invasion we used S. cerevisiae strain MY1560 (MATα/MATαΔ::LEU2 ura3Δ::P_{FLO11}-neo/ura3Δ0 leu2Δ0/leu2Δ0 trp1Δ0::hisG/trp1Δ10::hisG his3Δ0::hisG/his3Δ0::hisG). This strain contains the FLO11 promoter (P_{FLO11}) fused to the neo gene conferring resistance to the drug G418. FLO11 is a cell-surface flocculin required for pseudohyphal and invasive growth in S. cerevisiae. Cells exhibiting altered expression of FLO11 are expected to exhibit altered invasion ability. Thus, altered FLO11 expression can be used as an indicator of an altered invasion phenotype.

It was empirically determined that the wild type MY1560 strain (untransformed or transformed with a plasmid representing the peptide library staph nuclease scaffold) is unable to grow on YPD media supplemented with 100 μg/ml G418. In contrast, hyperinvasive strains are resistant to this concentration of the drug and form colonies on G418-supplemented YPD media. Thus, transformation of this strain with an invasion-promoting peptide should render the cells capable of growing on G418-supplemented YPD media.

Transformation of S. cerevisiae was by lithium acetate/single-stranded carrier DNA/polyethylene glycol (LiAc/ss-DNA/PEG) protocol (see, e.g., Gietz and Woods (1998), in: Methods in Microbiology, Chapter 26, Brown and Tuite, eds., Academic Press, New York; Agatep et al. (1998), TTO 1:P01525, Technical Tips Online at http://tto.trends.com) with a library of plasmids expressing unique random peptide sequences in the context of a Staphylococcal nuclease “scaffold” to generate more than 20 million independent Ura⁺ transformants (Norman et al. (1999), Science 285:591-595). The transformants were replica-plated to YPD plates supplemented with 100% g/ml G418 to select for clones with increased expression of the invasion-related reporter (FLO11) and consequent G418 resistance. Plates were incubated at 30° C. for 2-4 days and resistant clones were collected in pools (50 pools total) by scraping the colonies off each plate in a small volume of water. Plasmids were isolated as a pool (Hoffman and Winston (1986), Gene 57:267), transformed into E. coli, and re-transformed as a pool into the original selection strain, MY1560. Ura⁺ transformants were again replica plated to YPD+100 μg/ml G418 plates. Isolated colonies (“primary picks”) were re-streaked from each G418 plate that exhibited enrichment (i.e., more G418-resistant colonies than obtained when transforming with vector backbone (pTCN23) alone). Positive clones were further tested for plasmid dependence by (1) examining the correlation between spontaneous loss of the peptide-expressing plasmid and loss of G418 resistance, and (2) recovering the plasmid from the yeast, retransforming into the original selection strain, and characterizing the G418 resistance. Clones that failed to show plasmid dependence were eliminated from further study. The remaining candidate peptides were characterized for their effects on expression of P_{FLO11}-lacZ reporter constructs. This was performed by co-transforming the candidate plasmid into S. cerevisiae strain MY1405 (MATα ura3Δ0 his3Δ0::hisG trp1Δ0::hisG) which contains plasmid MB1339 [P_{FLO11}-lacZ TRP1 CEN] and measuring β-galactosidase activity. Plasmids that did not significantly affect expression of either reporter were eliminated from further analysis.

Candidate plasmids were further characterized to determine whether they expressed a peptide that modulated invasion using a standard invasion assay against controls (see, e.g., Roberts and Fink (1994), Genes Dev. 8(24):2974-85) on YPD. Briefly, candidate plasmids were transformed into S. cerevisiae strain MY1560 and two transformants from each were patched onto SC-Ura (synthetic complete media lacking uracil) adjacent to control cells expressing either the parent nuclease scaffold without a random peptide insert (negative control) or cells over-expressing FLO8 (an invasion-promoting gene and hyperinvasive control; contained on plasmid MB500). Cells were grown for two days at 30° C., replica-printed to YPD and grown for three days at 30° C. The YPD plates were held under a gentle stream of tap water for two minutes to wash off non-invading cells. The remaining invading cells are expected to harbor plasmids encoding bioactive peptides that promote the expression of FLO11.

### EXAMPLE 2

Selection of Peptides that Modulate Expression of SMP1 and Inhibit Invasion.

SMP1 is transcription factor of the MADs box family whose expression is inversely correlated with pH signaling and the invasion response. Cells exhibiting altered expression of SMP1 are expected to have altered invasion ability. Thus, altered SMP1 expression can be used a an indicator of an altered invasion phenotype.

To select for random peptides that increased SMP1 expression, the SMP1 promoter (P_{SMP1}) was fused to the neo gene to generate plasmids MB1037 [P_{SMP1}-neo URA3 CEN] and MB1337 [P_{SMP1}-neo TRP1 CEN]. It was empirically determined that wild type strains (e.g., S. cerevisiae strain MY295 or MY1406) were unable to grow on YPD media supplemented with as little as 50 μg/ml of G418. In contrast, hypoinvasive strains (e.g., rim101 mutants) form colonies on media supplemented with up to 200 μg/ml of the drug. Thus, transformation of this strain with an invasion-inhibiting peptide should render the cells capable of growing on G418-supplemented YPD media.

S. cerevisiae strain MY1561 (MATα/MATαΔ::LEU2 ura3Δ::P_{SMP1}-neo/ura3Δ0 leu2Δ0/leu2Δ0 trp1Δ0::hisG/trp1Δ0::hisG/his3Δ0::hisG/his3Δ0::hisG) was transformed with the peptide library as described above to generate 10 million independent Ura⁺ clones. The transformed clones were mated to lawns of S. cerevisiae strain MY1406 [MB1337] and subsequently replica-plated to SC-Ura-Trp plates to select for triploids carrying the library clones in addition to the P_{SMP1}-neo reporter. (It had been previously determined that the P_{SMP1}-neo construct borne by S. cerevisiae strain MY1561 was incapable of expressing G418 resistance, and therefore the episomal version was introduced by conjugation, as described above). The MY1561/MY1406 [MB1337] triploids were replica-plated to YPD plates containing 100, 150, and 200 μg/ml G418, and resistant colonies were picked and retested, and their phenotypes were checked for library plasmid dependence using 5-FOA. Promising candidates were lysed and their plasmids recovered in E. coli, amplified, and reintroduced into MY1561 along with MB 1337(MY1561 is an S. cerevisiae strain and MB 1337 is a plasmid). The drug-resistant phenotype was reconfirmed and shown to depend on the presence of MB1337. In addition, S. cerevisiae strain MY1405 was transformed with the peptide clones along with MB1338 [P_{SMP1}-lacZ TRP1 2 micron] and MB1339 [P_{FLO11}-lacZ TRP1 2 micron], and the enzyme assays were monitored. Seven peptide inhibitors of invasion were identified and are summarized in Table 2. Each of these peptides inhibits invasion in a standard invasion assay relative to a control strain. Similarly, each of the invasion-inhibiting peptides increases expression of SMP1 (as determined by increased G418 resistance or β-galactosidase activity in appropriate reporter strains) and decreases expression of FLO11 (as determined by decreased β-galactosidase activity). Peptide inhibitors of invasion were further characterized for their effects on calcofluor resistance, a phenotype associated with mutations in the rim101/pH response pathway. Expression of these peptides increases resistance to calcofluor, mimicking the phenotype associated with loss of function mutations in the rim101/pH response pathway. These peptides represent candidate genes for modulating fungal physiology for purposes including modulating, e.g., increasing or decreasing production of secondary metabolites and blocking pathogenesis.

### EXAMPLE 3

Binning of P_{FLO11} and P_{SMP1} Peptides Based on Effects on P_{FLO11} Promoter Fragments

Using a 96-well format of a lithium acetate transformation protocol (Elble (1992), Biotechniques 13:18-20), S. cerevisiae strain S. cerevisiae strain MY1406 (MATα, ura3Δ0, leu2Δ0, his3Δ0:hisG, trp1Δ0:hisG) was transformed with a collection of TRP1-marked plasmid-based FLO11::lacZ reporter constructs (see, e.g., Rupp et al. (1999), EMBO J 18:1257-1269) and grown on SC-Trp. The collection of reporter constructs consisted of P_{FLO11} deletion and fragment elements. Deletion elements consisted of fourteen serial 200 bp P_{FLO11} deletions that spanned the region 2800 bp upstream of the FLO11 initiation codon. Fragment elements consisted of fourteen individual 400 bp P_{FLO11} fragments, overlapping by 200 bp.

Using the same transformation protocol cited above, S. cerevisiae strain MY1407 (MAT α, ura3Δ0, his3Δ0:hisG, trp1Δ0:hisG) was transformed with plasmids directing the over-expression of invasion-promoting or invasion-inhibiting peptides and was grown on SC-Ura to select for peptide-expressing transformants. To serve as a control in this experiment, the staph nuclease-expressing parent strain, pTCN23, was transformed into MY1407 and grown on SC-Ura.

Peptide-expressing MATα regulator strains were mated to the panel of MATα FLO11:lacZ reporter strains in 96-well plates and diploids were selected by spotting onto SC-Ura-Trp plates.

The modulating effect of each candidate peptide upon each P_{FLO11} deletion or promoter fragment was quantitated using a 96-well lacZ assay. lacZ induction was measured spectrophotometrically and reported in Miller units. Ratios were calculated by dividing the lacZ induction produced by a particular peptide on a particular FLO11:lacZ reporter by the lacZ induction produced on the same FLO11:lacZ reporter by the staph nuclease parent, pTCN23.

### EXAMPLE 4

Identification of Peptides that Modulate the Hyphal Transition in C. albicans.

Next, the ability of a invasion-promoting polypeptide to promote invasion in a different species, C. albicans, was tested. Briefly, C. albicans strain MC258 (ura3Δ::λimm434/ura3Δ::λimm434, his1::hisG/his1::hisG, arg4::hisG/arg4::hisG, ade2::URA3:pHWP1-lacZ/ADE2) was transformed with Nru1-digested MB927 to generate a heterozygous HIS1 (his1::hisG/HIS1) strain. Control peptide pTCN23 and invasion-promoting peptide 5-32,1 (a peptide from S. cerevisiae, Table 1) were each cloned into an ARG+ C. albicans expression plasmid to generate MB2755 and MB2756, respectively. These plasmids were restricted with Asc1, and the cut DNA was used to transform HIS1+-modified MC258 (lithium acetate protocol). Transformants were selected on YNB plates (2 days at 30° C.), and several transformants from each transformation plate were tested by streaking onto deoxygalactose plates to identify those transformants for which the plasmid DNA had integrated properly at the GAL locus (properly integrated transformants exhibit a papillating phenotype on deoxygalactose).

To assess the modulating effect of peptide 5-32,1 on the hyphal transition relative to the pTCN23 control peptide, a serum induction assay was performed. C. albicans transformed with properly integrated and peptide-expressing pTCN23 or 5-32,1 were inoculated into YNB/2% glucose/pH 4.5 (2 mL) and grown overnight at 30° C. 1:10 dilutions of the overnight cultures were measured spectrophotometrically (OD₆₀₀). 4.5×10⁷ cells of each sample were collected, pelleted (1600 rpm, 2 minutes) and resuspended in inducing media (1 mL each: 0.5 mL 2× YNB, 100 μl filtered bovine calf serum, 0.05 mL 40% glucose, 0.05 mL 1 M MES pH 4.5, 0.3 mL water). Samples were incubated with aeration at 37° C., and aliquots were taken at 30 minute intervals and examined under a microscope. Photographs to document morphology differences between pTCN23 and 5-32,1-expressing cells were taken and the numbers of germ-tube and yeast-form cells were scored for each timepoint examined.

### EXAMPLE 5

Invasion-Promoting Peptides.

A large number of invasion-promoting peptides (>30) were identified using the methods of the invention. A description of 26 of these peptides is disclosed in Table 1. Importantly, each of the peptides caused increased invasion when expressed in S. cerevisiae (relative to a control strain) as determined in a standard invasion assay as well as by increased expression of FLO11 (as measured by increased G418 resistance or β-galactosidase activity in appropriate reporter strains). These peptides are candidates for modulating fungal physiology for purposes including modulating production of enzymes, primary and secondary metabolites and altering pathogenesis. The genetic targets of these invasion-promoting peptides may be regulators of pathogenesis

### EXAMPLE 6

Peptides that Exhibit S. cerevisiae/C. albicans Cross-Species Bioactivity.

The regulatory pathways in S. cerevisiae that impact invasion are known to be evolutionarily conserved and important for secondary metabolite production and virulence in filamentous and pathogenic fungi, respectively. Invasion-modulating peptides may function by binding a protein target that has an impact on the invasion phenotype. Enzyme active sites tend to be the most highly conserved regions of protein structure within a family of functionally related proteins and, therefore, peptide modulators of invasion are expected to exhibit cross-species function when tested for related phenotypes in other fungi.

One invasion-promoting peptide, 5-32,1, was assessed for a cross-species effect on hyphal transition in the pathogenic fungus C. albicans using a serum induction assay. C. albicans transformants expressing either control peptide pTCN23 or invasion-promoting peptide 5-32,1 were inoculated into YNB/2% glucose/pH 4.5 and grown overnight at 30° C. 4.5×1 cells were pelleted, resuspended into inducing media, and incubated at 37° C. Peptide 5-32,1 was found to be effective in inducing the morphological switch between yeast and filamentous growth relative to cells expressing the parent staph nuclease scaffold. The effect of expressing this peptide suggests the presence of negative regulators of the hyphal transition in C. albicans, that if knocked out, would render cells hyperfilamentous. Most important is the fact that the effect of this peptide on the hyphal transition validates the target of the peptide as capable of being functionally blocked by a small molecule. Peptides showing similar cross-species phenotypes can serve as valuable tools for the regulation of fungal physiology as well as for the acceleration of drug discovery.

### EXAMPLE 7

Invasion-Promoting and Inhibiting Peptides Interact with Multiple Signaling Pathways.

The FLO11 promoter has complex regulatory structure and integrates the signaling of a number of regulatory pathways that impact the invasion response. A series of FLO11::lacZ reporter constructs (consisting of either serial 200 bp deletions or 400 bp fragments across the span of the FLO11 promoter) was used to help determine how many different invasion-related signaling pathways can be targeted by the peptides. We examined the effect of each peptide on different regions of the FLO11 promoter and used that data to construct a functional fingerprint for each peptide. Peptides which have a common mechanism of action are expected to have related or identical fingerprints. A Pearson algorithm was used to group peptides having similar fingerprints and to determine similarity distances between these groups.

Significantly, three peptides identified in the P_{SMP1}-neo genetic selection that were tested against the FLO11-lacZ panel of reporters clustered together and had inhibitory effects on lacZ induction relative to the parent staph nuclease (pTCN23). With regard to peptide activators of P_{FLO11}, seven of the most potent peptides grouped into two different categories that activate different regions of the FLO11 promoter. These results suggest that the peptides identified in the P_{FLO11}-neo and P_{SMP1}-neo genetic selections target different proteins residing in different signaling pathways.

Forty-two peptide activators of P_{FLO11} and three peptide activators of P_{SMP1} were assessed for their effects on a series of FLO11-lacZ reporter constructs in S. cerevisiae. In this study, lacZ ratios were generated by dividing the lacZ induction produced by a particular peptide on a particular FLO11:lacZ reporter by the lacZ induction produced on the same FLO11:lacZ reporter by the staph nuclease parent, pTCN23. Cluster analysis of the ratios was performed using single linkage and Pearson distance.

### EXAMPLE 8

### Identification of Bioactive Peptides that Modulate lacZ Expression in C. albicans.

Control peptide pTCN23 (MB2755) and invasion-promoting peptide 5-32,1 (MB2756) are restricted with Asc1, and the digested DNA are used to transform C. albicans strain MC297 (ura3Δ::λimm434/ura3Δ::λimm434, his1::hisG/his1::hisG, arg4::hisG/arg4::hisG, HWP1::HWP1p-lacZ(URA3). Transformants are selected on media lacking arginine (2 days at 30° C.), and several transformants from each transformation plate are tested by streaking onto deoxygalactose plates to identify those transformants for which the plasmid DNA has integrated properly at the GAL locus (properly integrated transformants give a papillating phenotype on deoxygalactose).

To assess the modulating effect of peptide 5-32,1 on the P_{HWP1}-lacZ reporter relative to the pTCN23 control peptide, visual screens are carried out by patching colonies onto X-Gal plates. A slightly modified X-Gal recipe, SMM, is more sensitive for detection of β-galactosidase. SMM contains 1.7 g Yeast Nitrogen Base (without amino acids or ammonium sulfate), 20 g glucose, 5 g ammonium sulfate and 20 g agar in 930 mL water. After autoclaving, 70 mL 1 M potassium phosphate pH 7.0 and 2 mL of a 20 mg/mL X-Gal solution is added.

β-galactosidase activity is noticeable in regions of the streak that appear wrinkled and in the center of the streak; these regions contain a higher percentage of filamentous cells than the edge of the streak which contains C. albicans primarily in the budding yeast form. A comparison of staph nuclease-expressing cells to those cells expressing peptide 5-32,1 reveals a higher level of β-galactosidase expression for the cells expressing peptide 5-32,1.

TABLE 1
Bioactive Peptide Sequences
(*denotes a stop codon)

| | |
|---|---|
| GRRSRVRWSWPLFKSL | (SEQ ID NO. 1) |
| QPWYRKLRLAPVYPSD | (SEQ ID NO. 2) |
| PYGPFLLLTPF*A*L | (SEQ ID NO. 3) |
| ELAYKFGRDWARLYFS | (SEQ ID NO. 4) |
| KRKVCLLGGRFLVEWL | (SEQ ID NO. 5) |
| RRWEVLRGDRTARLLS | (SEQ ID NO. 6) |
| ALARWLEIELHPQGLI | (SEQ ID NO. 7) |
| AFSRRGRRAWSWPVSN | (SEQ ID NO. 8) |
| SGDAVMGFLLKCLGLQ | (SEQ ID NO. 9) |
| GHLWEWSGERWLLRWC | (SEQ ID NO. 10) |
| GYLRKHRALALGLNLH | (SEQ ID NO. 11) |
| YEAGVILRACLWAVSG | (SEQ ID NO. 12) |
| RCPVAHTLCWEVAGCT | (SEQ ID NO. 13) |
| CGLGGCCCEIFWSFSN | (SEQ ID NO. 14) |
| REINWLRFFRLHKVID | (SEQ ID NO. 15) |
| LYRAFTWPVAKSLRME | (SEQ ID NO. 16) |
| WWRPHERFIWAQYYLA | (SEQ ID NO. 17) |
| EWCEICHRLVWVFCFT | (SEQ ID NO. 18) |
| QRIPLIGGVLFIAWIM | (SEQ ID NO. 19) |
| LDLDRVSVNRCSFMGF | (SEQ ID NO. 20) |
| RRWNRLKSWWPCFREQ | (SEQ ID NO. 21) |
| HGDGLMRYFWSVAMWT | (SEQ ID NO. 22) |
| GAGCAPRGCVVWSSLL | (SEQ ID NO. 23) |
| GSNGCVSVSSSVTSFM | (SEQ ID NO. 24) |
| KRGRRRSHSCPSLMTD | (SEQ ID NO. 25) |
| MGCVKRLLWGCCMKLT | (SEQ ID NO. 26) |

TABLE 2
Bioactive Peptide Sequences

| | |
|---|---|
| LGYMRASREMDGWKLL | (SEQ ID NO. 27) |
| LPYEVSRVCWPRRLGY | (SEQ ID NO. 28) |
| LGYGEAEREMVTCREH | (SEQ ID NO. 29) |
| LAYGPERYPRACMRWD | (SEQ ID NO. 30) |
| LSYMEGPYWHRSFRAV | (SEQ ID NO. 31) |
| LTGYAKLLVQLSYFAM | (SEQ ID NO. 32) |
| LPVSYVGQSTSQGPVW | (SEQ ID NO. 33) |

What is claimed is:

1. A method for identifying cross-species bioactive peptides, comprising:
providing cells of a first species having a first phenotype;
transforming the cells of a first species having a first phenotype with a nucleic acid molecule encoding a polypeptide comprising a random peptide sequence to provide a library of cells of the first species expressing a polypeptide comprising a random peptide sequence;
selecting from the library of cells of the first species a cell having a second phenotype;
identifying the random peptide sequence expressed by the selected cell as a bioactive polypeptide;
expressing the bioactive polypeptide in a cell of a second species having a first phenotype to produce a cell of a second species having the second phenotype; and identifying a bioactive peptide that when expressed in the cell of the second species produces a cell of the second species having a second phenotype as a cross-species bioactive peptide;

2. The method of claim 1 wherein the first and second species are selected from the group consisting of mammalian species.

3. The method of claim 1 wherein the first and second species are selected from the group consisting of mammalian species and fungal species.

4. The method of claim 1 wherein the first and second species are fungal species.

5. The method of claim 1 wherein the polypeptide does not comprise either a DNA binding domain or a transcription activation domain.

6. The method of claim 1 wherein the first species is selected from the group consisting of Saccharomyces cerevisiae, Aspergillus nidulans, Candida sp., and Neurospora crassa.

7. The method of claim 1 wherein the second species is selected from the group consisting of Candida sp., Aspergillus sp., Penicillium sp., Acremonium chrysogenum, Yarrowia lipolytica, Phaffia rhodozyma, Mucor sp., Rhizopus sp., Fusarium sp., Penicillium marneffei, Microsporum sp., Cryptococcus neoformans, Pneumocystis carinii, Trichophyton sp., and Ustilago maydis, Nodulisporium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Neurospora sp., Pestalotiopsis sp. and Phaffia rhodozyma.

8. The method of claim 1 wherein the cells of the first species are wild-type.

9. The method of claim 3 wherein the second phenotype of the first fungal species is selected from the group consisting of modulated expression of S. cerevisiae FLO11 or SMP1, modulated invasion, modulated colony morphology, modulated adherence to solid substrate, modulated pseudohyphal growth, modulated expression of a FLO11 reporter gene, modulated expression of a SMP1 reporter gene, conditional cell death, and inhibition of cell growth.

10. The method of claim 3 wherein the second phenotype of the second fungal species is selected from the group consisting of altered virulence, avirulent, and hypervirulent.

11. The method of claim 3 wherein the second phenotype of the second fungal species is selected from the group consisting of modulated production of a metabolite or enzyme, increased storage viability, altered cell or colony morphology, altered temperature tolerance, cell death, growth inhibition, conditional lysis, resistance to deleterious effects of exposure to a primary or secondary metabolite or enzyme, hypervirulence, avirulence, increased pathogenesis, decreased pathogenesis, altered adherence characteristics, and modulated expression of an HWP1-reporter gene

12. An isolated cross-species bioactive peptide selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, and SEQ ID NO. 26.

13. A method for identifying a genetic target responsible for fungal virulence, the method comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the first or second phenotype of the second fungal species.

14. An isolated cross-species bioactive peptide selected from the group consisting of SEQ ID NO. 27, SEQ ID NO. 28, SEQ ID NO. 29, SEQ ID NO. 30, SEQ ID NO. 31, SEQ ID NO. 32, and SEQ ID NO. 33.

15. A method for identifying a genetic target responsible for modulating primary metabolite production, the method comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the first or second phenotype of the second fungal species.

16. A method for identifying a genetic target responsible for modulating secondary metabolite production, the method comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the first or second phenotype of the second fungal species.

17. A method for identifying a genetic target responsible for enzyme production, the method comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the first or second phenotype of the second fungal species.

18. A method for identifying a genetic target responsible for a particular characteristic of a fungus, the method comprising expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype, choosing a fungus of the second species having the second phenotype, and identifying a target responsible for the first or second phenotype of the second fungal species, wherein the particular characteristic is selected from the group consisting of storage viability, altered cell morphology, altered colony morphology, temperature tolerance, cell death properties, growth inhibition, conditional lysis, and resistance to primary or secondary metabolite toxicity.

19. An isolated cross-species bioactive peptide identified by expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype.

20. A fungal cell expressing a cross-species bioactive peptide, said bioactive peptide identified by expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype.

21. A method for producing a primary metabolite, the method comprising culturing the fungus according to claim 20 under conditions suitable for production of the primary metabolite.

22. A method for producing a secondary metabolite, the method comprising culturing the fungus according to claim 20 under conditions suitable for production of the secondary metabolite.

23. A method for producing an enzyme, the method comprising culturing the fungus according to claim 20 under conditions suitable for production of the enzyme.

24. Peptidomimetics of a bioactive peptide identified by expressing in a first fungal species having a first phenotype a bioactive peptide to yield a second phenotype, choosing a fungus of the first species having the second phenotype, identifying the bioactive peptide in the chosen fungus of the first fungal species, expressing the bioactive peptide in a second fungal species having a first phenotype to yield a second phenotype.

25. A method for identifying a cross-species peptide-interacting polypeptide that binds to a cross-species bioactive peptide, comprising:
providing cells of a first species having a first phenotype;
transforming the cells of a first species having a first phenotype with a nucleic acid molecule encoding a polypeptide comprising a random peptide sequence to provide a library of cells of the first species expressing a polypeptide comprising a random peptide sequence;
selecting from the library of cells of the first species a cell having a second phenotype;
identifying the random peptide sequence expressed by the selected cell as a bioactive polypeptide;
expressing the bioactive polypeptide in a cell of a second species having a first phenotype to produce a cell of a second species having the second phenotype;
identifying a bioactive peptide that when expressed in the cell of the second species produces a cell of the second species having a second phenotype as a cross-species bioactive peptide;
contacting the cross-species bioactive peptide with a test polypeptide to determine if the test polypeptide interacts with the cross-species bioactive peptide; and identifying the test polypeptide as a cross-species peptide-interacting polypeptide if the test polypeptide binds to the cross-species bioactive peptide.

26. The method of claim 21 wherein the polypeptide expressed by the cells of the library are identical except for the random polypeptide sequence.

27. The method of claim 21 wherein the random polypeptide sequence comprises between 5 and 50 amino acids.

28. The method of claim 21 wherein the random polypeptide sequence comprises between 5 and 40 amino acids.

29. The method of claim 21 wherein the random polypeptide sequence comprises between 10 and 30 amino acids.

30. The method of claim 21 wherein the random polypeptide sequence comprises between 10 and 30 amino acids.
